(19) United States
(12) Patent Application Publication
(10) Pub. No.: US 2024/0252805 A1
(43) Pub. Date: Aug. 1, 2024

(54) CONNECTION INTERFACE FOR STERILE CONNECTION AND FLUID TRANSFER

(71) Applicants: Geoffrey L. Hodge, Sutton, MA (US); SOTIO BIOTECH INC., Boston, MA (US)

(72) Inventors: Geoffrey L. Hodge, Sutton, MA (US); Daniel S. Kline, Boston, MA (US); Michael W. Maccollum, Boston, MA (US); Scott W. Beaver, Boston, MA (US)

(21) Appl. No.: 18/565,456

(22) PCT Filed: Jun. 1, 2022
(86) PCT No.: PCT/US2022/031763

§ 371 Date: Nov. 29, 2023

Related U.S. Application Data
(60) Provisional application No. 63/196,020, filed on Jun. 2, 2021.

(30) Foreign Application Priority Data
Aug. 26, 2021 (EP) 21193321.3

(51) Int. Cl. A61M 39/18; A61M 39/10

(52) U.S. Cl. CPC A61M 39/18; A61M 2039/1072

(57) ABSTRACT

The disclosure includes a system and method for making a sterile connection for transferring a liquid between two containers, including: (a) positioning a first connection surface and a second connection surface; (b) coupling the first connector and the second connector, wherein the coupling defines a sterilization chamber comprising a gap between the first connection surface and the second connection surface; (c) activating a sterilization agent within the sterilization chamber to produce a sterile environment (d) fluidically coupling the first container with the second container through the first connection surface and the second connection surface, (e) transferring a liquid between the first container and the second container.

### CROSS-REFERENCE TO RELATED APPLICATIONS

This application claims the benefit of, and priority to, U.S. Provisional Application No. 63/196,020, titled “Connection Interface for Sterile Connection and Fluid Transfer”, filed on Jun. 2, 2021, and European Patent Application No. EP21193321.3, titled “Connection Interface for Sterile Connection and Fluid Transfer”, filed on Aug. 26, 2021, the disclosures of each of which are incorporated by reference herein in their entirety.

### FIELD

This application relates to a connection interface for sterile connection and fluid transfer, e.g., during manufacturing of cells.

### BACKGROUND

Adoptive cell therapy, e.g., chimeric antigen receptor (CAR)-T cell-based therapy, is becoming a promising option for treating various types of cancer because of its potential to evade genetic and cellular mechanisms of drug resistance, and to target tumor cells while sparing normal tissues. Efficient manufacturing of high-quality therapeutic cells for clinical or commercial use is an opportunity for the wide application of this technology.

Current approaches for producing therapeutic cells for use in adoptive cell therapy typically involve ex vivo enrichment, activation, and expansion of T cells and genetic modification of the T cells using retroviral or lentiviral vectors to introduce an exogenous nucleic acid coding for a chimeric receptor. This whole process is time consuming and expensive. It is of great interest to develop devices and methods that enable efficient, high-volume, and cost-effective production of therapeutic cells.

### SUMMARY

Provided herein are connection interfaces for sterile connection and liquid transfer and methods of use thereof. Connection interfaces provided herein may be used for sterile liquid transfer during manufacturing of cells to reduce cross-contamination and increase manufacturing productivity and efficiency.

In a first embodiment, the disclosure relates to a method for making a sterile connection for transferring a liquid between two containers, including: (a) positioning a first connection surface and a second connection surface, wherein: the first connection surface is linked to a first container and the second connection surface is linked to a second container, (b) coupling the first connector and the second connector, wherein the coupling defines a sterilization chamber comprising a gap between the first connection surface and the second connection surface; (c) activating a sterilization agent within the sterilization chamber to produce a sterile environment (d) fluidically coupling the first container with the second container through the first connection surface and the second connection surface, (e) transferring a liquid between the first container and the second container, and (f) optionally disconnecting the first connection surface and the second connection surface after step (e).

In a second embodiment, the disclosure relates to a device comprising: holders for positioning a first connection surface and a second connection surface, wherein: the first connection surface is linked to a first container and the second connection surface is linked to a second container, the first connector and the second connector define a sterilization chamber comprising a gap between the first connection surface and the second connection surface, a pump configured to transfer a liquid from the first container to the second container, and a sterilization agent that is activatable within the gap between the first connection surface and the second connection surface.

In a third embodiment, the disclosure relates to a connection interface for sterile connection and liquid transfer, includes: a first connector and a second connector. The connection interface also includes a first sterilizable space, the first connector and the second connector define the sterilization space between the first connection surface and the second connection surface, wherein the sterilizable space, such as the gap, can comprises a sterilization agent. The first connector is fluidically coupled with a first container and the second connector is fluidically coupled with a second container in the first sterilizable space, and the connection interface includes a liquid transfer device including pumps and valves to allow for liquid transfer between the first container and the second container and/to avoid back contamination.

In a fourth embodiment, a first connector coupled with a second connector to define an enclosed sterilization chamber around the connectors.

### BRIEF DESCRIPTION OF THE DRAWINGS

Various aspects and embodiments will be described with reference to the following figures. The figures are not necessarily drawn to scale.

FIGS. 1A-1B are schematic depictions of an exemplary connection interface, according to some embodiments. 100—connection assembly, 110—first container, 120—connector, 130—housing, 140—sterilizable space, 150—second container, 160—connector, 170—fluid conduit, 180—sterilizer, 190—valve. FIG. 1A: without sterilizer. FIG. 1B: with sterilizer.

FIGS. 2A-2F are schematic depictions of an exemplary connection interface, in accordance with some embodiments of the technology described herein. 200—connection assembly, 170a, 170b—fluid conduit, 190, 190a, 190b—valves, 200a, 200b—connectors, 210 intermediate piece, 230—seals, 240a, 240b—spaces, 250a, 250b—ports. FIGS. 2A and 2B: schematic depictions of two connectors before (2A) and after connection (2B). FIGS. 2C—2E: schematic depictions of an exemplary connection interface before connection (2C), after connection (2D), and fluid flow through after connection (2E). FIG. 2F: a schematic depiction of an exemplary connection interface in a sterilizing chamber.

FIGS. 3A-3F are schematic depictions of an exemplary process for sterile connection and liquid transfer, in accordance with some embodiments of the technology described herein, including load component (3A), sterilize components (3B), make connection (3C), transfer liquids (FIG. 3D), break connection (3E), and eject components (3F).

FIGS. 4A-4B are schematic depictions of an exemplary process for manufacturing cells, in accordance with some embodiments of the technology described herein. FIG. 4A: an exemplary process for cell culture. FIG. 4B: an exemplary process for manufacturing cells transduced with a viral vector.

FIG. 5 is a flowchart illustrating steps in a method for making a sterile connection for transferring a liquid between two containers, according to some embodiments.

### DETAILED DESCRIPTION

The present disclosure is based, at least in part, on the development of connection interfaces and use thereof for performing sterile connections and liquid transfers during, for example, processes of manufacturing cells for therapeutic use. The connection interfaces disclosed herein allow for transferring liquid between a first container and a second container, e.g. a cell culture vessel via a connector in a sterile manner. The connection interfaces and methods using such disclosed technology herein led to at least the following advantageous outcomes:

A) High-throughput production of manufactured cells resulting from connection interfaces configured for use during various stages of the manufacturing process, e.g., for use during cell expansion and/or cell transduction. High-throughput production may also be achieved from connection interfaces disclosed herein that minimize the time needed to perform multiple liquid transfers. The connection interfaces disclosed herein also are easy to automate, thereby increasing production rates and productivity for processes that require multiple serial connections.

B) Reduced negative effects of sterilization on cell growth. Connection interfaces disclosed herein provide sterilization at or near the connection using a sterilization technique that is shallow in nature, which minimizes diffusion of sterilizing agents into the cell culture where they may harm the cells.

C) Reduced risk of cross contamination between containers and/or cell culture vessels resulting from connection interfaces configured for use with connectors that may be sterilized between uses or disposed of after a single use.

Accordingly, provided herein are connection interfaces for sterile connection and liquid transfer and methods of use thereof. Such connection interfaces may be used for sterile connection and liquid transfer during cell manufacturing, e.g., manufacturing cell therapies such as T cells expressing a chimeric antigen receptor (CAR).

### I. Connection Interfaces for Sterile Connection and Liquid Transfer

The present disclosure provides a connection interface for connecting a first container and a second container, optionally a cell culture vessel via a connection interface in a sterilizable space, and transferring a liquid between the first container and the second container thus connected.

Accordingly, a connection interface disclosed herein may include one or more connectors and one or more sterilizable spaces, wherein the one or more connectors are operable to connect to two or more containers and/or a cell culture vessel in the sterilizable spaces. In some examples, the connection interface may further include one or more containers, which may be cell culture vessels, cell culture bags and/or a container for fluids, for example media or solutions of choice to be added. A container, as used herein, refers to any container suitable for holding a solution or suspension. A connector, as used herein, refers to an apparatus that is arranged to connect the containers in a sterilizable space.

Any sterilizable space may be suitable for connecting the container(s) and the cell culture vessel via the connector. In some examples, a connection interface may include one or more sterilizable spaces. For example, the connection interface may include a housing that forms a sterilizable space for performing a sterile connection and liquid transfer. Alternatively, or in addition to, connectors optionally with an intermediate piece may form a sterilizable space. For example, the connection interface may include two or more pieces that form one or more sterilizable spaces for performing a sterile connection and liquid transfer. In some embodiments, the two or more pieces may be located in a housing that forms one or more sterilizable spaces. In some embodiments, the first connector or the second connector comprise a first piece and a second piece, wherein the first piece and the second piece form the first sterilizable space, a second sterilizable space, and/or a third sterilizable space; or the first connector or the second connector comprise a first piece and a second piece, the first piece and the second piece comprise one or more valves, one or more seals, and one or more ports.

In some examples, the sterilizable space may include a sterilizer and/or one or more ports operable to receive a source of the sterilizer for sterilization. Portions of the fluid conduit to the container(s), and the connectors with the optional intermediate piece may be sterilized in the sterilizable space in the housing of the connection interface using the sterilizer.

In an embodiment, the disclosure relates to a connection interface for sterile connection and liquid transfer, wherein the connection interface comprises a first connector, a second connector, wherein the first connector and the second connector define a sterilization chamber comprising a gap between the first connection surface and the second connection surface, wherein the gap is an enclosed space accessible through at least one opening, for example a port, wherein the gap optionally comprises a sterilization agent wherein the first connector is fluidically coupled with a first container and the second connector is fluidically coupled with a second container and a liquid transfer device including pumps and valves to allow for liquid transfer between the first container and the second container and/to avoid back contamination. In some instances, the gap between the first connection surface and the second connection surface is via partial coupling wherein the gap is a closed space accessible through a port.

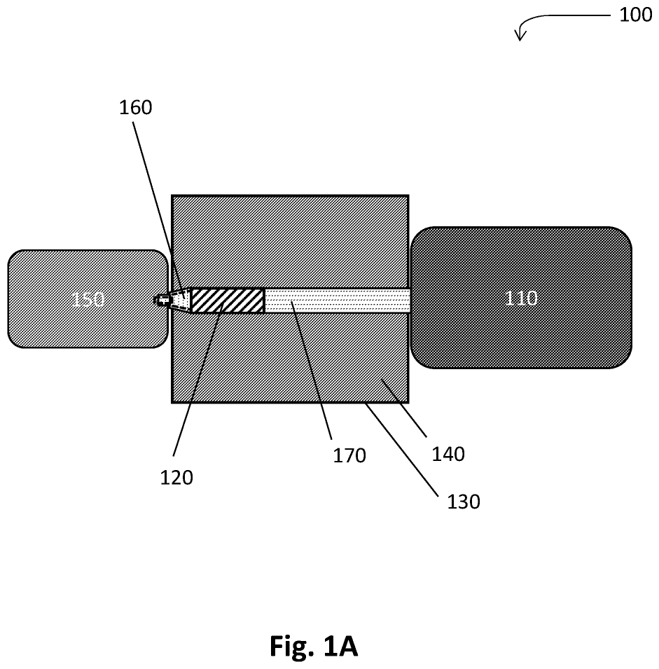

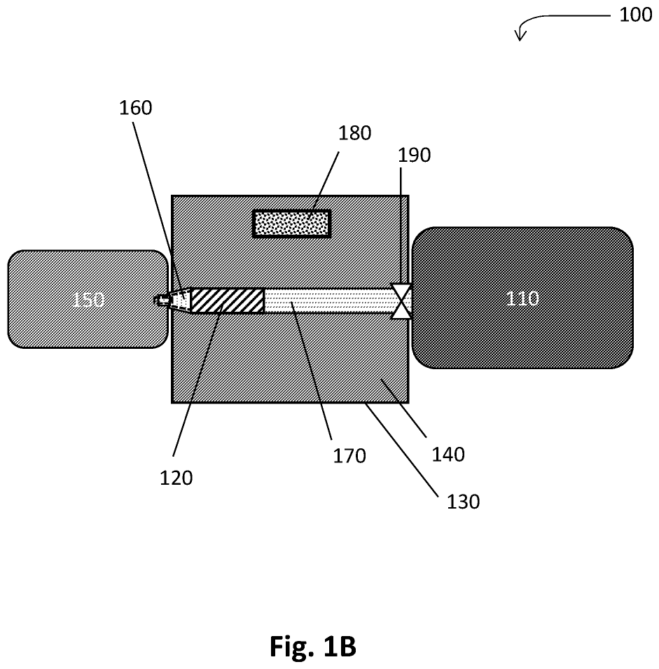

FIGS. 1A-1B are schematic depictions of an exemplary connection assembly 100 including a container 110, a connector 120, and a housing 130 forming a space 140 for sterile connection and liquid transfer between a first container 110 and a second container 150 via the connector 120, in accordance with some embodiments of the technology described herein. FIG. 1A shows the connector 120 attached to a connector 160 of the container 150 and a fluid conduit 170 of the container 110. FIG. 1B shows the connection interface of FIG. 1A further including a sterilizer 180, and a container 110 that further includes a valve 190.

FIG. 1A shows a connection assembly 100 that may include a connector 120 and connector 160 in a housing 130 that forms a sterilizable space 140 for sterile connection and liquid transfer between a container 110 and a second container 150. The connectors may removably connected to the first container 110 and the second container 150. The connector 120 may be attached to the container 110 and the cell culture vessel 150 in any suitable manner. For example, connector 120 is fluidly attached to a fluid conduit 170 of the container 110 and to a connector 160 of the second container 150. In some examples, the connection assembly 100 may include a container 110, wherein the container may be a cell culture vessel. In one embodiment, connectors 120 and 160 mechanically interlock to form a fluid path. In another embodiment, connector 120 comprises a cannula and connector 160 comprises a septum. In an embodiment, the connection interface for sterile connection and liquid transfer comprises the first container includes a solution for transferring into a cell culture vessel. In other embodiments, the second container comprises a cell culture vessel. In some embodiments a solution in the first container is a culture medium for culturing cells grown in the second container.

FIG. 1B shows the connection interface that may include a valve or other suitable arrangement for controlling liquid flow and/or discouraging backflow. The valve may be a one-way valve for allowing fluid flow in a single direction or a bidirectional valve for allowing fluid flow in either direction. The valve may optionally be a pinch valve external to flexible tubing. The fluid conduit 170 of the container 110 may include a valve 190 (e.g., a check valve) to control the flow of liquid and discourage backflow. The valve may be located in any suitable position, e.g., at a proximal end of the respective fluid conduit. It will be appreciated that other suitable arrangements may be utilized to encourage fluid flow in the desired direction. For example, the connection interface may include interlocked process controls. In another example, the connection interface may further include a pump operable to pump the contents between the first container and the second container, in either direction. In some embodiments, the connection interface further comprises a pump for liquid transfer between the second container and the first container, a second container, and/or the third container. For example, a pump may start just before opening a valve, creating positive or negative pressure to assure immediate flow of fluid in the desired direction.

The connection interface may include a sterilizer for sterilizing the sterilizable space and components placed within that space. Housing 130 may include a sterilizer 180 that sterilizes the sterilizable space 140 and components of the connection interface placed within the sterilizable space 140, including the connector 120 and portions of the first container 110 and second container 150. Sterilization may be performed before connecting the first container to second container via connectors 120 and 160 and/or after disconnecting the first container and the second container.

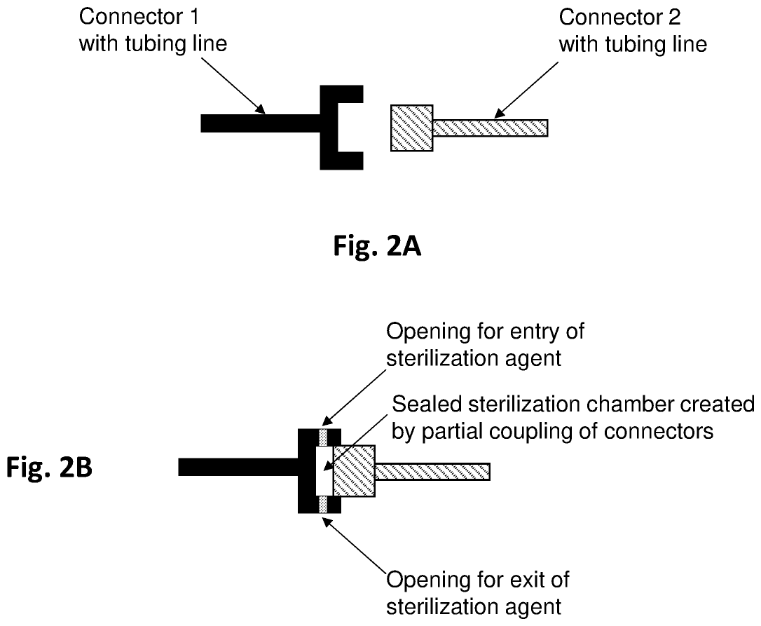

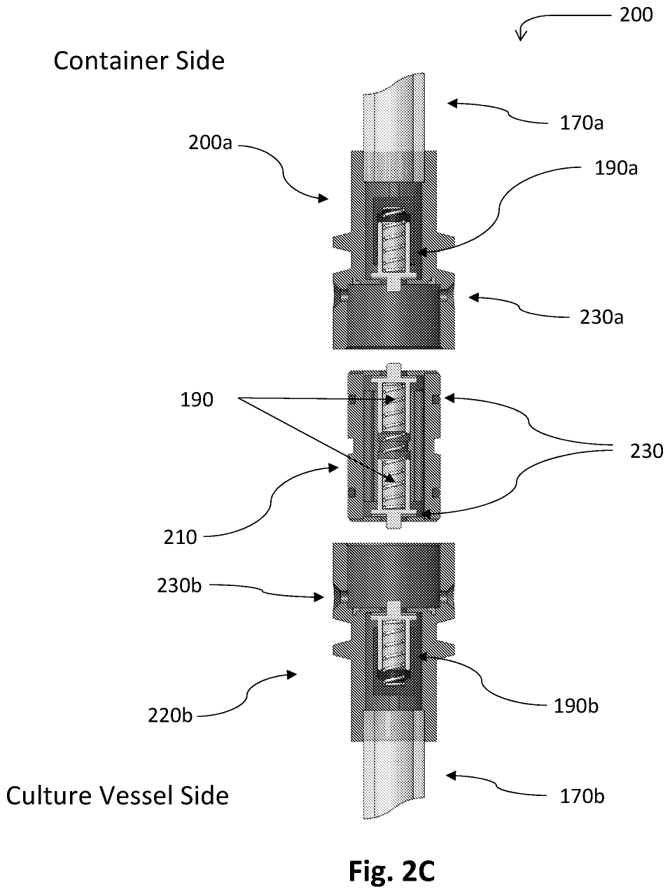

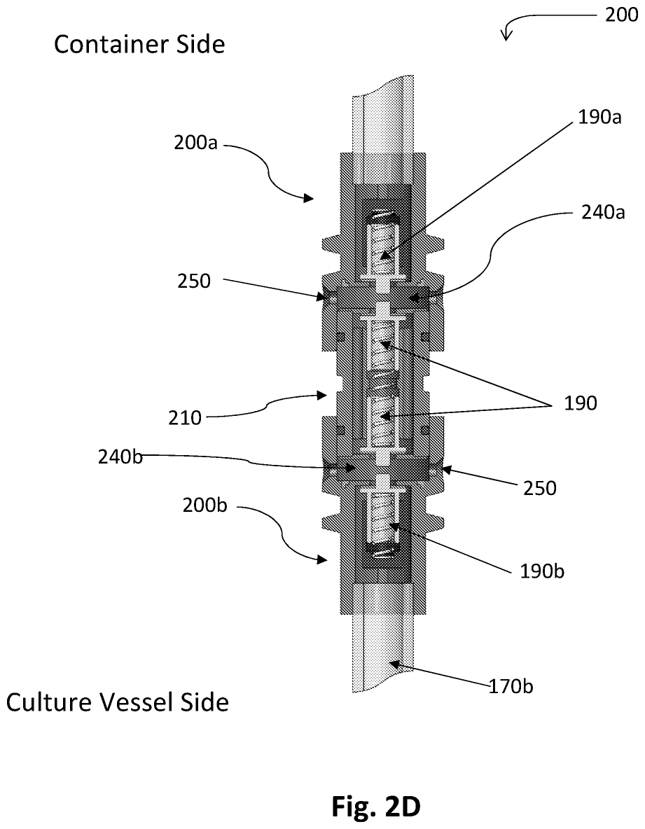

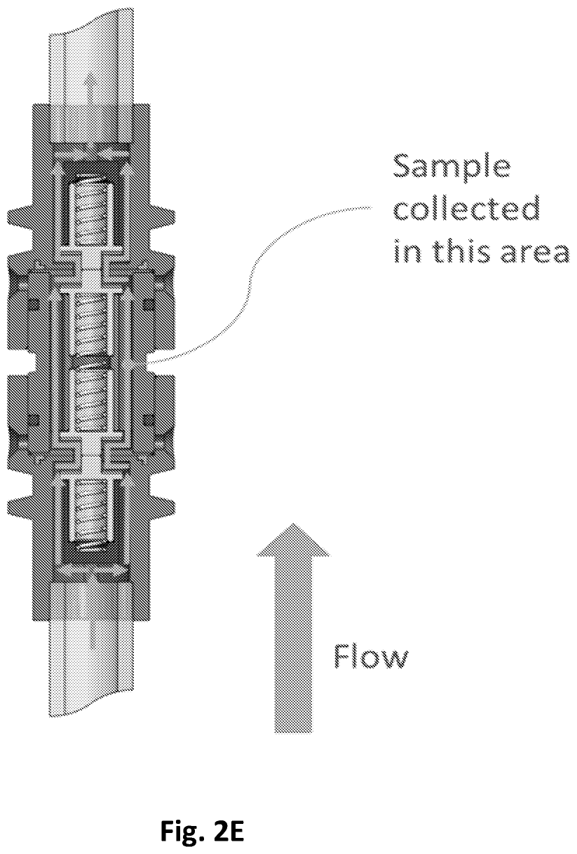

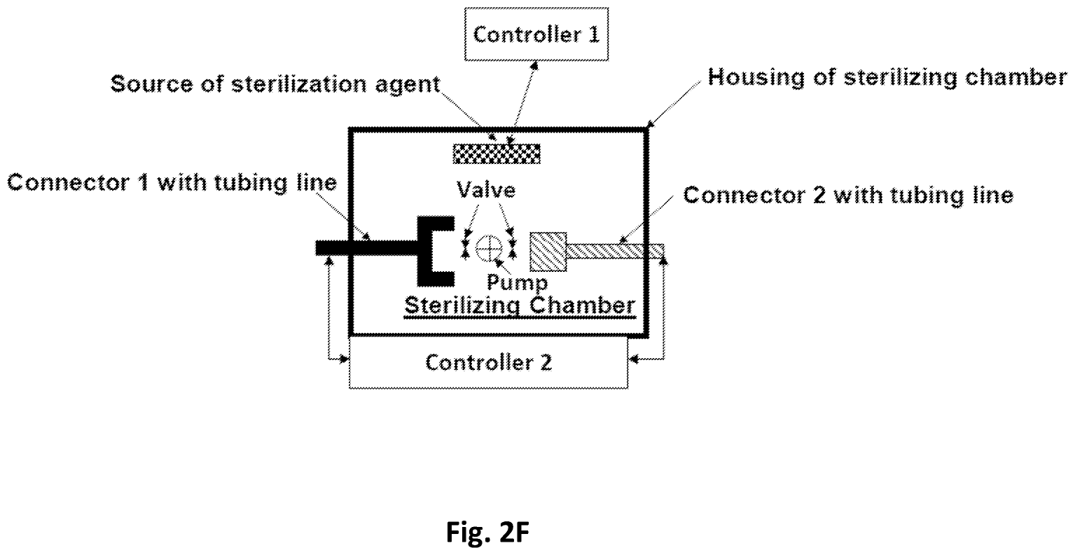

FIGS. 2A-2D are schematic depictions of exemplary connectors and connector interfaces, in accordance with some embodiments of the technology described herein.

FIG. 2A is a simplified version of an engineering schematic of designed fittings. Accordingly, the first connector (including a tubing line) and the second connector (including a tubing line) may be configured to form a sealed sterilization chamber by partially coupling the connectors. A first opening for entry of the sterilization agent and a second opening for exit of the sterilization agent are formed in the sealed sterilization chamber. In some embodiments, the sterilization agent comprises a fluid, a gas, or a vapor. Accordingly, a fluid sterilization agent may flow through the sealed sterilization chamber to effectively sterilize the connection interface. The fluid sterilization agent may be a gas, a liquid, or a hot vapor (e.g., water), and the like.

FIG. 2C shows the connection assembly 200 with the connector 200a attached to the fluid conduit 170a of the first container, connector 200b attached to the fluid conduit 170b of the second container, and an intermediate piece 210. In some embodiments, the connectors 200a and 200b may be arranged to form one or more sterilizable spaces. The intermediate piece 210 may removably connect to the connector 200a and the connector 200b. The connector 200a may include a valve 190a and a seal 230a, the intermediate piece 210 may include two valves 190 and two seals 230, and the connector 200b may include a valve 190b and a seal 230b. The valves 190 may be operable to control the flow of fluid through the connector. The seals 230 may be operable to provide fluid and airtight seals between the connector 200a and the intermediate piece 210 and between the connector 200b and the intermediate piece 210. In another example, the two valves 190 is eliminated and the intermediate piece 210 is connected to the fluid conduit 170b, creating only a single sterilizable space. In one embodiment, valves 190 and 190a are configured to comprise opposing pins which, upon compression of the connector 200a and the intermediate piece 210, meet each other and, against the springs within the valves, open the flow path (FIG. 2E).

FIG. 2D shows additional spaces 240a and 240b for sterile connection and liquid transfer formed from attaching each end of the intermediate piece 210 to connectors 200a and 200b. The additional space 240a and 240b may be sterilized via ports 250a and 250b (hereinafter, collectively referred to as “ports 250”) in the connectors. In some embodiments, one or more sterilizable spaces 240a and 240b are formed by coupling each end of the intermediate piece 210 to the first and second connector 200a and 200b. The sterilizable space 240a and 240b may be sterilized via ports 250 in the first and second connectors 200a and 200b. For example, the ports 250 may be operable to receive a source of steam for sterilizing the sterilizable space 240a between the connector 200a and the intermediate piece 210 and the sterilizable space 240b between the connector 200b and the intermediate piece 210. The sterilization spaces formed by partial compression of the connectors 200a and 200b are maintained by spring-held valves 190, 190a and 190b at the connection surface, obstructing the sterilizing agent from the fluid flow path (170a and 170b).

FIG. 2E shows the connectors 200a (aka 120), 200b (aka 160), and intermediate piece 201, fully engaged to allow fluid transfer. The opposing pins of spring valves, e.g. 190, 190a and 190b, are pushed open by compression of the connectors 200a and 200b to unseal the fluid flow path. This figure notes that the seals 230a, 230b of the intermediate piece 201, when broken, will contain fluid between the seals and therefore can be used for sample removal.

FIG. 2F shows a connection interface, according to some embodiments. The connection interface includes a sterilizable chamber within a housing. The sterilizable chamber is configured to receive a first connector fluidically coupled to a first container and a second connector fluidically coupled to a second container. In some embodiments, a sterilization agent in the first sterilizable chamber is activated to sterilize the first connector and the second connector. In some embodiments, the connection interface may also include a pump configured to transfer a liquid from the first container to the second container. In some embodiments, the connection interface may include an interlock valve configured to avoid back contamination between the first container and the second container. In some embodiments, the pump may be a peristaltic pump, including one or more interlocking pinch valves between the first container and the second container to prevent backflow between the first container and the second container. In some embodiments the pump and/or pinch valves may be located inside the sterilizable space. In other embodiments the pump and/or pinch valves may be located outside the sterilizable space.

A connector may be removable and/or disposable and/or reusable. The connector may be removed (or disconnected) from a container, e.g. a cell culture vessel, after performing a sterile connection and liquid transfer. Any suitable manner may be used to remove the connector from the connection interface, e.g., by ejecting the connector from the connection interface. A connector may be disposable such that a new connector may be used for each sterile connection and liquid transfer between a first container and a second container. A connector may be reusable such that it may be used in multiple sterile connections and liquid transfers. In such instances, the connector may be sterilized prior to each use. In some instances, there may be multiple connectors. In some embodiments, the first connector, the second connector, and/or the third connector is removable, disposable, reusable, or a combination thereof. In some embodiments, the first connector, the second connector, and/or the third connector is ejectable from the connection interface.

A connector may include one or more parts, e.g., one or more cannulas and/or one or more septae, and/or one or more mechanical fittings and/or one or more pieces of tubing. For example, when the connector includes a single piece of tubing, one end of the tubing is arranged to be welded to a fluid conduit of a container and the other end of the tubing is arranged to be welded to a fluid conduit of a cell culture vessel. In another example, when the connector includes multiple pieces of tubing, an intermediate portion of tubing may be used to connect two or more pieces of tubing. In some instances, multiple connectors are connected. In exemplary embodiments, the first connector, and/or the second connector each comprise a first piece and a second piece, one end of the first piece being arranged to be attached to the first container, a second container, and/or the third container and one end of the second piece being arranged to be attached to the second container. In some embodiments, the first connector, the second connector, and/or the third connector comprises a septum and/or a cannula.

An optional intermediate piece may be removable and/or disposable. Alternatively, or in addition to, the intermediate piece may be configured for liquid sampling through one or more ports. In some embodiments, the first connector or the second connector comprise a first piece and a second piece, further comprising an intermediate piece having a first end and a second end, which are arranged to be attached to a second end of the first piece and/or a second end of the second piece.

A connector may include one or more features useful for sterile connection and liquid transfer. For example, a connector may include one or more valves to control the flow of liquid. In another example, a connector may include one or more seals to prevent leakage. In yet another example, a connector may include one or more ports to allow access to a sterilizing agent, e.g., steam.

A container for use in a connection interface disclosed herein may be any suitable shape or size, and any suitable material. For example, when receiving cells in the cell culture, the container may be a gas permeable material that permits diffusion of gases sufficient for cell viability. Such containers may be suitable for processing the cells, e.g., culturing and/or centrifuging the cells in the container. Alternatively, or in addition to, the container may be disposable to eliminate risks of contamination. Non-limiting examples of a container include a cell culture container (e.g., a cell culture bag or a cell culture flask), a destination bag, a source bag, vial, or syringe (e.g., for adding liquids like media, viral vector suspension or solutions of growth hormones, cytokines, drugs, or other compounds to manipulate cells) or a waste container. A destination bag may be used for either receiving the cell culture medium or the cells of the cell culture.

A container for use in a connection interface disclosed herein may include a fluid conduit and/or opening for transferring liquid between the first container and the second container. For example, a container may include a fluid conduit, which may be attached to a connector when performing a sterile liquid transfer. Alternatively, or in addition to, a container may include a cannula, a fitting for receiving a cannula, or a septum for transferring liquid under sterile conditions.

A container for use in a connection interface disclosed herein may be any suitable shape or size, and any suitable material. For example, a container may be a cell culture vessel that is disposable to eliminate risks of contamination. A non-limiting example of a cell culture vessel includes a cell culture bag and/or a rigid cell culture vessel with a vent and/or gas permeable membrane.

A container for use in a connection interface disclosed herein may include a fluid conduit and/or opening for transferring liquid between a first container, a second container, a third container and so forth. For example, the container may include a fluid conduit, which may be attached to a connector when performing a sterile liquid transfer. Alternatively, or in addition to, the container may include an opening including a septum for transferring liquid under sterile conditions.

Liquid may be transferred between the first container and the second container in either direction. In some embodiments, liquid may be transferred from the first container to the second container. In some embodiments, liquid may be transferred to the first container from the second container. The first container may be empty to receive the contents of the second container or the first container may include a solution to be transferred into the second container. In some embodiments, the first container, the second container, and/or the third container comprises a fluid conduit, and the first connector, the second connector, and/or the third connector is arranged to be attached to the fluid conduit. In some embodiments, the second container comprises a septum, and the first connector, the second connector, and/or the third connector is arranged to be attached to the septum. In other embodiments, the first connector or the second connector each comprise a first piece and a second piece, the first container and the second container comprise a fluid conduit, and the first piece is arranged to be attached to the fluid conduit, and the second container comprises a septum, and the second piece is arranged to be attached to the septum.

A non-limiting example of a solution to be transferred from the container to the cell culture vessel is a culture medium for culturing cells in the cell culture vessel. Alternatively, or in addition to, the solution includes a nucleic acid for transducing cells grown in the cell culture vessel. Such nucleic acids may be delivered into cells using conventional technologies, e.g., transduction using reagents such as liposomes or viral transduction (e.g., retroviral transduction such as lentiviral transduction). When the connection interface is being used to manufacture cells expressing a chimeric antigen receptor (CAR), the solution may include a nucleic acid encoding the CAR. In specific embodiments, a solution in the first container comprises a nucleic acid or a viral particle comprising such for transducing cells grown in the second container, and wherein the nucleic acid encodes a chimeric receptor (for e.g. CAR).

Any suitable number of containers and/or connectors may be included in a connection interface disclosed herein. In some embodiments, a connection interface may include one or more containers and two or more connectors for sterile connection and liquid transfer between a cell culture vessel and the one or more containers via the one or more connectors. In such instances, the one or more containers and the cell culture vessel may be connected via the two or more connectors in the same sterilizable space or in different sterilizable spaces.

In some embodiments, the connection interface disclosed herein may comprise multiple containers, one being the source container and the others being the destination containers. A liquid (e.g., cell culture medium) can be transferred between contains in a one-to-many manner, e.g., from the source container to each of the destination containers (e.g., containing cells) in a sequential manner via serial connections and disconnections.

In some embodiments, the connection interface disclosed herein may comprise multiple containers, one being the destination container (e.g., containing cells) and the others being the source containers (containing culture medium, viral vectors, growth factors, etc.). A liquid can be transferred in a many-to-one manner between containers, e.g., from each of the multiple source containers to the destination container in a sequential manner via serial connections and disconnections.

In some embodiments, the connection interface disclosed herein may comprise multiple containers, some of which are destination container (e.g., containing cells) and the others are source containers (containing culture medium, viral vectors, growth factors, etc.). A liquid can be transferred in a many-to-many manner between containers, e.g., from each of the multiple source containers to each of the multiple destination container in a sequential manner via serial connections and disconnections.

In some embodiments, a connection interface may include a first and a second container and a first and a second connector for sterile connection and liquid transfer between the cell culture vessel and the first container via the first connector, and between the cell culture vessel and the second container via the second connector. In such instances, the first container may include a cell culture medium for transferring into the cell culture vessel, which includes the cell culture, and the second container may be a destination bag for receiving either the culture medium or the cells of the cell culture. In other embodiments, the second container comprises a destination bag for receiving either a culture medium or multiple cells from a cell culture.

In some embodiments, a connection interface may include a first, a second, and a third container and a first, a second, and a third connector for sterile connection and liquid transfer. This setup can be used for liquid transfer between a cell culture vessel and the first container (e.g. containing media) via the first connector, between the cell culture vessel and the second container (e.g. containing waste) via the second connector. In a further embodiment this setup can be easily extended with a fourth container (e.g. containing a suspension with a non-viral or viral vector for cell transduction) and a forth connector between the cell culture vessel and the forth container, or can be extended with multiple further pairs of containers and connectors for e.g. adding solutions comprising compounds for cell manipulation. Similarly, in some embodiments, a connection interface may include multiple pairs of containers being cell culture vessels with different cell cultures and connectors, and one or more pairs of containers and connectors for media, and/or solutions/suspensions for cell manipulation. In exemplary embodiments, the first container comprises a cell culture medium or a viral vector for transferring into the second container, which comprises a first cell culture, wherein the connection interface further comprises a third container including a second cell culture configured to receive the cell culture medium or the viral vector from the first container.

A connection interface disclosed herein may further include a housing. The housing may surround and/or contain the connector and one or more sterilizable spaces. In some embodiments, the housing forms a sterilizable space. A housing may be any suitable shape or size, and any suitable material.

A housing may surround one or more connectors or portions thereof. For example, the housing may surround a first connector and second connector. In another example, the housing may surround a first connector, a second connector, and a third connector.

A housing may contain one or more connectors or portions thereof. For example, the housing may contain a first connector and second connector. In another example, the housing may contain a first connector, a second connector, and a third connector.

A housing may surround one or more containers or portions thereof. Alternatively, or in addition to, the housing may surround one or more cell culture vessels or portions thereof. For example, the housing may surround a fluid conduit of a container and/or a fluid conduit of a cell culture vessel. In another example, the housing may surround a fluid conduit of a container and a septum of a cell culture vessel.

A housing may contain one or more containers or portions thereof. Alternatively, or in addition to, the housing may contain one or more cell culture vessels or portions thereof. For example, the housing may contain a fluid conduit of a container and/or a fluid conduit of a cell culture vessel. In another example, the housing may contain a fluid conduit of a container and a septum of a cell culture vessel.

A connection interface disclosed herein may further include a device that facilitates sterile connection and/or liquid transfer. In an exemplary embodiment, the disclosure provides a device comprising (i) holders for positioning a first connection surface and a second connection surface, wherein the first connection surface is integral to a first connector and the second connection surface is integral to a second connection; and wherein the first connection surface is linked to a first container and the second connection surface is linked to a second container, (ii) the first connector and the second connector configured to form a sterilization chamber comprising a gap between the first connection surface and the second connection surface; (iii) a pump configured to transfer a liquid from the first container to the second container and (iv) a sterilization agent that is activatable within the gap between the first connection surface and the second connection surface, wherein the gap is an enclosed space accessible through at least one opening. In an embodiment, the first connector and/or the second connector comprises ports suitable to provide access for the sterilization agent to the gap. In some embodiments, the device comprises the first connector and the second connector configured to form, via partial coupling, a gap between the first connection surface and the second connection surface.

For example, the connection interface may include a device for sterile connection such as a sterilizer. The sterilizer may include an energy source that directs energy towards the sterilizable space and components placed within that space. The energy source can be heat and/or steam. Alternatively or additionally, the sterilizer may be a sterilizer agent, such as a fluid selected from a gas (e.g., ozone), a sterilizing chemical (e.g., ethanol) or a vapor. In some embodiments, the device may include an interlock valve (for e.g., pinch valve) configured to avoid back contamination between the first container and the second container. In other embodiments, the device, comprises one or more interlocking pinch valve between the first container and the second container to prevent backflow between the first container and the second container.

In some embodiments, the pump may be a peristaltic pump, including one or more interlocking pinch valves between the first container and the second container to prevent backflow between the first container and the second container. In some embodiments, a peristaltic pump between the first connector and the second connector to prevent backflow between the first container and the second container.

In some embodiments, the device may include a first controller configured to activate the sterilization agent over the first connector and the second connector. In some embodiments, a second controller configured to position the first connector and the second connector for sterilization and/or to remove the first connector or the second connector from the sterilization position after liquid transfer between the first container and the second container. In an embodiment, the device contains a controller configured to automatically operate any of the steps or in combination.

In another example, the connection interface may include a device for liquid transfer such as a pump, a vacuum, or a pressurizer. For example, a pump may be connected to the connector, the fluid conduit of the container, and/or the fluid conduit of the cell culture vessel. Alternatively, or in addition to, the connection interface may use gravity to facilitate liquid transfer. Any of the devices for sterile connection and/or liquid transfer may be located in the housing of the connection interface.

It should be appreciated that various embodiments of the present disclosure may be formed with one or more of the above-described features. The above aspects and features of the disclosure may be employed in any suitable combination as the present disclosure is not limited in this respect. It should be appreciated that the drawings illustrate various components and features which may be incorporated into various embodiments of the present disclosure. For simplification, some of the drawings may illustrate more than one optional feature or component. However, the present disclosure is not limited to the specific embodiments disclosed in the drawings. It should be recognized that the present disclosure encompasses embodiments which may include only a portion of the components illustrated in anyone drawing figure, and/or may also encompass embodiments combining components illustrated in different figures.

### II. Methods for Sterile Connection and Liquid Transfer

Also provided herein are methods for making a sterile connection for transferring a liquid between at least two containers using a connection interface disclosed herein. Such methods involve at least three steps comprising (a) positioning a first connection surface and a second connection surface, wherein the first connection surface is integral to a first connector and the second connection surface is integral to a second connector; and wherein the first connection surface is linked to a first container and the second connection surface is linked to a second container; (b) activating a sterilization agent over the first connection surface and the second connection surface within a sterilization chamber to produce a sterile environment; (c) fluidically coupling the first container with the second container through the first connection surface and the second connection surface; (d) transferring a liquid between the first container and the second container; and optionally (e) disconnecting the first connection surface and the second connection surface after step (d), wherein step (a) comprises forming a gap between the first connection surface and the second connection surface via partial coupling of the first and second connectors, and wherein step (b) comprises activating the sterilization agent within the gap.

In an exemplary embodiment, a method for making a sterile connection for transferring a liquid between at least two containers, comprises (a) positioning a first connection surface and a second connection surface, wherein the first connection surface is integral to a first connector and the-second connection surface is integral to a second connector; and wherein the first connection surface is linked to a first container and the second connection surface is linked to a second container; (b) coupling the first connector and the second connector, wherein the coupling defines a sterilization chamber comprising a gap between the first connection surface and the second connection surface; (c) activating a sterilization agent within the sterilization chamber to produce a sterile environment; (d) fluidically coupling the first container with the second container through the first connection surface and the second connection surface; (e) transferring a liquid between the first container and the second container; and optionally (f) disconnecting the first connection surface and the second connection surface after step (e).

In one embodiment, the connection surfaces and connectors are placed inside a sterilization chamber, wherein a sterilization agent is present in or introduced to the chamber to sterilize the chamber, the connectors, and the connection surfaces.

In another embodiment, the connectors are partially engaged, with a gap between the connection surfaces, forming a small sterilization chamber within the connectors. Sterilization agent is introduced to the gap between the connection surfaces to sterilize the connection surfaces, prior to the connectors being fully engaged to form a fluid flow path. In an embodiment, the gap between the connection surfaces is an enclosed space accessible through at least one opening, such as a port. Suitable connectors are shown in FIG. 2. The sterilization agent can enter and exit the gap through the ports. Also, ports can easily be controlled in an automated fashion so that the sterilization step can be automated. Accordingly, in an embodiment, the sterilization agent is a fluid, a gas or steam suitable for sterilization that can quickly be routed through the ports into the gap and across the connection surfaces.

In other embodiments, activating the sterilization agent (for e.g., fluid, gas, vapor, or steam) comprises flowing the sterilization agent through the gap between the connection surfaces through the ports. In an embodiment, steam is used under high pressure at temperatures of about 120° C. to 140° ° C. for about 5 minutes and at least 2 bar for sterilization. Suitable gases and fluids for sterilization are well known in the art, e.g., suitable gases are ethylene oxide or ozone. A suitable fluid may contain ethanol or peracetic acid.

In some embodiments, the first and second connectors are loaded into a housing, optionally wherein any of the steps (a)-(f) or in combination is operated automatically by a controller. In some embodiments, the method comprises the first and second connectors being loaded into a housing, optionally wherein positioning the first connection surface and the second connection surface in step (a) comprises robotically loading the first connector and the second connector within the housing. The configuration allows for an automated sterile connection and liquid transfer between containers, as all of the steps can be easily automated and sterilization of the gap and the connection surfaces prior to fluidically coupling of the containers is fast and efficient given the small volume that needs to be sterilized. Compared to configurations where a whole chamber comprising containers, tubings and connectors are sterilized, the configuration of this embodiment allows for fast sterilization of the gap and connection surfaces, whereas respective mounts required for automated movement of the tubings, containers and connectors with all their surfaces do not require new sterilization with every new connection.

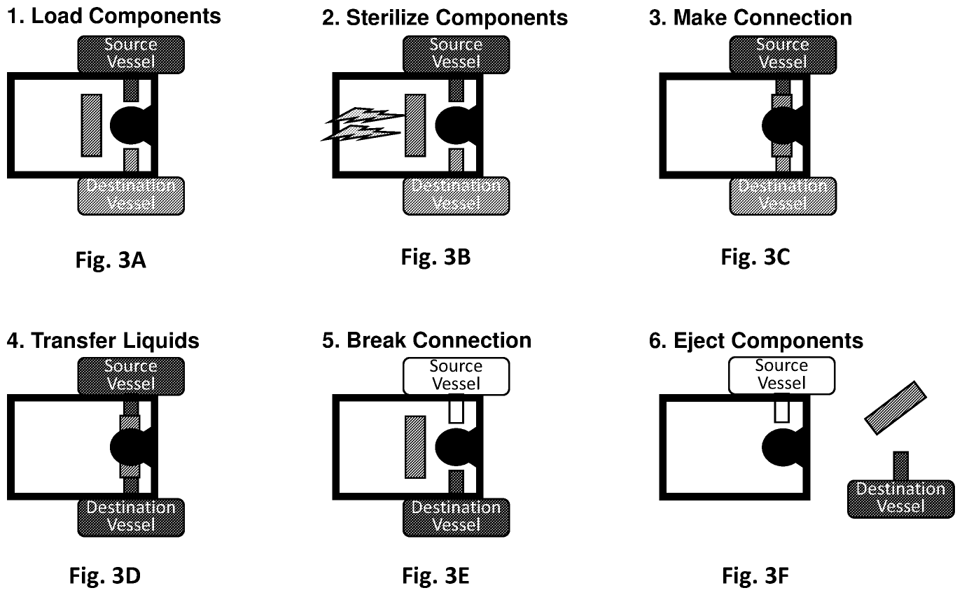

FIGS. 3A-3F are schematic depictions of an exemplary process for sterile connection and liquid transfer, in accordance with some embodiments of the technology described herein. The process may include an exemplary method for sterile connection and liquid transfer between a source vessel (e.g., a container) and a destination vessel (e.g., a cell culture vessel) using a connection interface disclosed herein.

FIG. 3A illustrates the connection interface being loaded with the source vessel, the destination vessel, and a connector (rectangle). In this illustrative example, the connection interface includes a pump (circle) to transfer liquid from the source vessel to the destination vessel.

FIG. 3B illustrates portions of the vessels (e.g., fluid conduits) and the connector that are then sterilized in the sterilizable space in the housing of the connection interface.

FIG. 3C illustrates that the source vessel is then connected to the destination vessel via the connector.

FIG. 3D shows liquid transfer from the source vessel to the destination vessel.

FIG. 3E illustrates that the source vessel and the destination vessel are disconnected from the connector, after the desired amount of liquid is transferred.

FIG. 3F illustrates that the connector and destination vessel are then ejected from the connection interface.

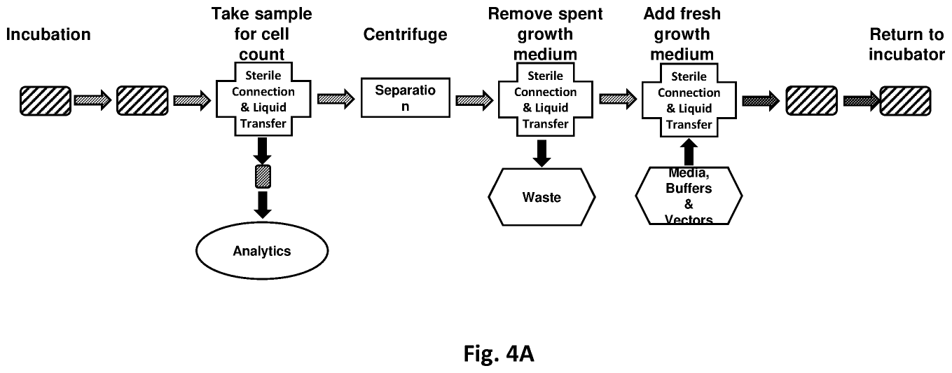

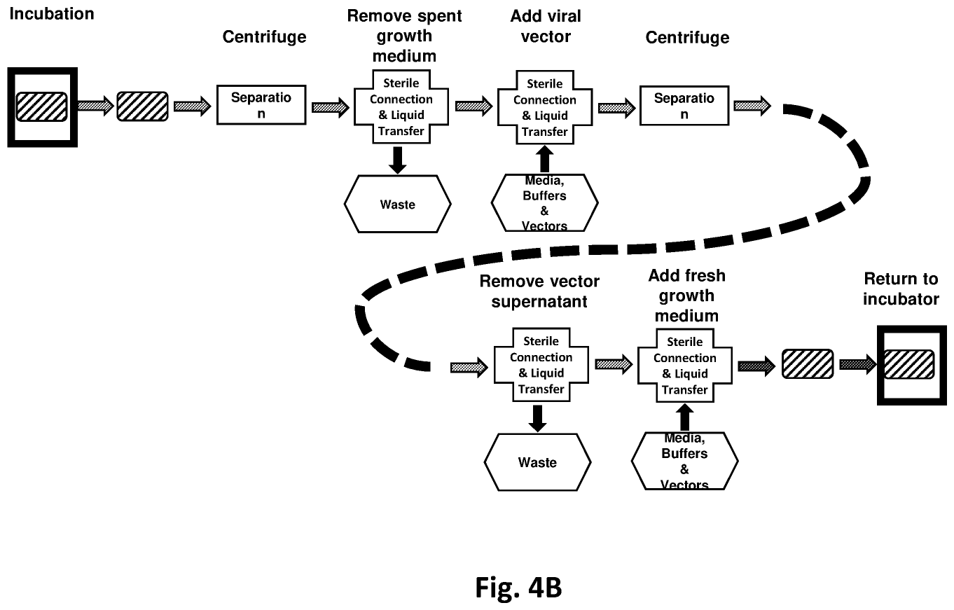

FIGS. 4A-4B are schematic depictions of an exemplary process for manufacturing cells, in accordance with some embodiments of the technology described herein.

FIG. 4A is a schematic depiction of an exemplary process for expanding a cell culture. A connection interface may be used to perform various liquid transfers involved in the cell expansion process including taking a sample of the cells for analysis, removing spent growth medium, and adding fresh medium. A device capable of performing serial sterile connection and liquid transfer operations is shown as the blue plus in the workflow of a typical cell culture manufacturing operation of cell growth medium addition. External steps may be performed manually, or containers may be transferred by robotic arms or similar automated transfer devices. Automated loading of the connectors, sterilization, connection, liquid transfer, and disconnection of containers are performed by the sterile connection and liquid transfer device. In this example, the sterile connection and liquid transfer device is used first to remove medium (from the cell culture source bag to a destination waste medium bag) then to add liquid from the cell culture medium source bag to destination cell culture bag). Note that the waste medium destination bag and cell culture medium source bag may be serially connected to many cell culture bags (acting first as source bags, then as destination bags).

FIG. 4B is a schematic depiction of an exemplary process for transducing a cell culture. A connection interface may be used to perform various liquid transfers involved in the cell transduction process including adding viral vector, removing vector supernatant, removing spent growth medium, and adding fresh medium. A device capable of performing serial sterile connection and liquid transfer operations is shown as the blue plus in the workflow of a typical CAR-T cell therapy manufacturing operation of viral vector transduction. External steps may be performed manually, or containers may be transferred by robotic arms or similar automated transfer devices. Automated loading of the connectors, sterilization, connection, liquid transfer, and disconnection of the containers are performed by the sterile connection and liquid transfer device. In this example, the sterile connection and liquid transfer device is used first to remove medium (from the cell culture source bag to a destination waste medium bag), then to add liquid from the viral vector source bag to destination cell culture bag.

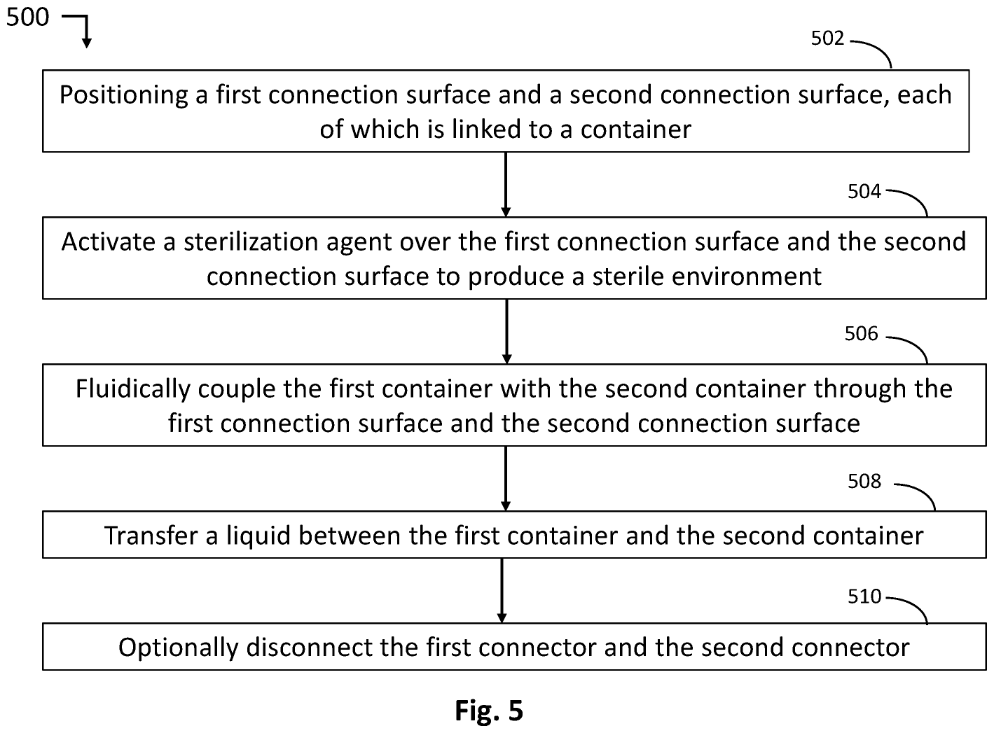

FIG. 5 is a flowchart illustrating steps in a method 500 for making a sterile connection for transferring a liquid between two containers, according to some embodiments. In some embodiments, method 500 may be performed using a device, as disclosed herein, the device including a first sterilizable chamber configured to receive a first connector fluidically coupled to a first container and a second connector fluidically coupled to a second container. The device may also include a pump configured to transfer a liquid from the first container to the second container, and a sterilization agent in the first sterilizable chamber. In some embodiments, method 500 may be performed in a connection interface for sterile connection and liquid transfer, as disclosed herein. The connection interface may include a first connector, a second connector, and a first sterilizable space, which optionally includes a sterilization agent. In some embodiments, at least one of the first connector or the second connector may be a re-usable connector. The first connector is fluidically coupled with a first container and the second connector is fluidically coupled with a second container in the first sterilizable space and a liquid transfer device including pumps and valves to allow for liquid transfer between the first container and the second container and to avoid back contamination. In some embodiments, the first container includes one or more source bags containing a culture medium or a waste medium, and the second container includes one or more culture bags containing a cell culture. In some embodiments, method for making a sterile connection for transferring a liquid between at least two containers, wherein the first container or the second container is a destination bag comprising a plurality of cells.

Especially for autologous pharmaceutical products multiple individual cell culture vessels may be incubated at the same time and may require sterile liquid transfer for cell manipulation or medium change at specific time, where automation may contribute to cost saving. Accordingly, in some embodiments, the second container comprises a cell culture vessel, and the method further comprising collecting a cell culture from an incubator housing a plurality of cell culture vessels. Accordingly, individual cell culture vessels can be taken from the incubator housing in an automated fashion to subject it to the method for making a sterile connection and liquid transfer according to the disclosure using the devises of the disclosure.

In some embodiments, the second container includes immune cells, or optionally T cells. In some embodiments, the second container includes immune cells, which optionally are T cells. Further, the immune cells can express a chimeric receptor. The provided methods are especially suitable for the automation of the manufacture of adoptive cell therapies given the multiple manufacturing steps of the cellular pharmaceutical products, which require multiple liquid transfers in a sterile fashion for manipulation and expansion of the cells.

Step 502 includes loading, within a chamber, a first connector and a second connector, wherein the first connector is linked to a first container and the second connector is linked to a second container, and the chamber includes a sterilization agent. In some embodiments, step 502 includes automated mechanical positioning the first connector and the second connector for sterilization. The first connector and the second connector are configured to establish a tight, hermetic connection. Optionally, the sterilization chamber is created by the partial engagement of two connectors such that the connection surfaces are not yet in contact creating a gap between the connection surfaces. The gap may be an enclosed space (240a and/or 240b) accessible through ports (250a and/or 250b).

Step 504 includes activating the sterilization agent over the first connector and the second connector to produce a sterile environment. In some embodiments, step 504 includes sterilizing previously non-sterile first connector and second connector. In some embodiments, the sterilization agent is a fluid, gas, vapor, heat or steam, and step 504 includes flowing the sterilization agent through the gap between connection surfaces, for example through ports into the closed space between the connection surfaces. The energy source is configured that the energy can sterilize the connection surfaces. In some embodiments, the sterilization agent is a fluid, and activating the sterilization agent includes flowing the fluid through at least a portion of the chamber. In some embodiments, activating the sterilization agent over the first connector and the second connector is performed prior to fluidically coupling the first container with the second container. In some embodiments, step 502 includes forming a gap between the first connector and the second connector, and step 504 includes activating the sterilization agent within the gap.

Step 506 includes fluidically coupling the first container with the second container through the first connector and the second connector. In some embodiments, wherein step 506 includes fluidically coupling the first and second container with a fluid transfer conduit, the fluid transfer conduit has connectors to couple either end of the fluid transfer conduit with the first connector and the second connector. In some embodiments, the coupling of the connectors and/or connection surfaces is achieved via mechanical automation.

Step 508 includes transferring a liquid between the first container and the second container. In some embodiments, the first container includes a cell culture medium and the second container includes cultured cells, and step 508 includes transferring the cell culture medium to the cultured cells. In some embodiments, the first container comprises a cell culture medium and the second container comprises cultured cells, and wherein transferring a liquid between the first container and the second container comprises transferring the cell culture medium to the cultured cells. In some embodiments, the first connector and the second connector are connected via a third connector such that the first connector connects to one end of the third connector and the second connector connects to the other end of the third connector, optionally wherein the intermediate connector is sealable for removal of samples from the fluid flow path. In some embodiments, step 508 includes interlocking one or more pinch valves and/or pumps. In some embodiments, transferring a liquid between the first container and the second container in step (d) comprises interlocking one or more pinch valves and/or pumps. In other embodiments, transferring a liquid between the first container and the second container in step (d) comprises rotating a peristaltic pump to cause a positive or a negative pressure in a tubing between the first container and the second container. In some embodiments, transferring a liquid between the first container and the second container in step (d) comprises rotating a peristaltic pump between two interlocking valves prior to activating the interlocking valves.

In some embodiments, step 508 includes rotating a peristaltic pump to cause a positive or a negative pressure in the tubing between the first container and the second container. In some embodiments, step 508 includes rotating a peristaltic pump between two interlocking valves prior to activating the interlocking valves. In some embodiments, step 508 includes transferring a solution including a nucleic acid or a viral particle including such, to the second container for transducing a plurality of cells therein, optionally wherein the nucleic acid encodes coding a chimeric receptor. In some embodiments, the first container is a cell culture vessel, the second container is a destination bag, and step 508 includes transferring a culture medium or a plurality of cells into the destination bag from a cell culture vessel. In some embodiments, the second container is a cell culture vessel, and step 508 includes connecting a third container and the cell culture vessel in the chamber, and transferring liquid between the cell culture vessel and the third container. In some embodiments, step 508 includes pumping the liquid from the first container to the second container. In some embodiments, the second container includes a cell culture vessel, and step 508 further includes collecting a cell culture from an incubator housing a plurality of cell culture vessels. Optionally, the method can be applied with multiple other containers.

Step 510 includes disconnecting the first connector and the second connector after step 508. In some embodiments, step 510 includes sealing a flow path between the first container and the second container. In some embodiments the disconnection is achieved via mechanical automation.

Methods disclosed herein involve sterile connection and liquid transfer between any source vessel and destination vessel. For example, when manufacturing cells, the source vessel may be a container including culture media and the destination vessel may be a cell culture vessel including a cell culture. In such instances, methods disclosed herein may be performed to transfer the culture media from the source vessel to the cell culture in the destination vessel.

A connector such as tubing may be used for collecting a sample of a liquid thus transferred. For example, the connector may have a portion suitable for collecting a sample in a portion of the connector, which may be referred to as a disposable intermediate. In some embodiments, when tubing is used as a connector, the tubing may be used for transferring liquid from the container to the cell culture vessel, and a portion of the tubing may be used to collect the liquid thus transferred. In another example, an additional connector may be used as a disposable intermediate. In some embodiments, when tubing is used as a connector, an additional piece of tubing may be used for transferring liquid from the cell culture vessel to the container, and the additional piece of tubing may be used to collect the liquid thus transferred. In each of these examples, the tubing is sealed, and the connection broken to create the disposable intermediate containing a sample of the liquid thus transferred. In such instances, the intermediate tubing also prevents backflow contamination between the first and second connector.

Any number of sterile connections and liquid transfers may be performed by repeating the moving step, the connecting step, and the transferring step until the desired number of sterile connections and liquid transfers has been performed. The desired number of sterile connections and liquid transfers can be performed using a single connection interface or multiple connection interfaces. A single connection interface may be used to perform a single sterile connection and liquid transfer or multiple sterile connections and liquid transfers. Alternatively, or in addition to, multiple sterile connections and liquid transfers may be performed using multiple connection interfaces.

Multiple sterile connections and liquid transfers to a cell culture vessel using a single connection interface may involve multiple pairs of one connector and one container each. In such instances, methods may include moving a cell culture vessel to a connection interface or vice versa, connecting a first connector to the cell culture vessel and a first container in the housing of the connection interface, transferring liquid between the cell culture vessel and the first container, and repeating the connecting and transferring steps using an additional connector and an additional container until the desired number of liquid transfers has been performed.

For example, when using a single connection interface for multiple sterile connections and liquid transfers, methods may include (a) moving a cell culture vessel to a connection interface or vice versa, (b) connecting the first connector to the cell culture vessel and the first container in the housing of the connection interface, (c) transferring liquid between the cell culture vessel and the first container, (d) connecting the second connector to the cell culture vessel and the first container in the housing of the connection interface, (e) transferring liquid between the cell culture vessel and the second container, (f) connecting the third connector to the cell culture vessel and the third container in the housing of the connection interface, and (g) transferring liquid between the cell culture vessel and the third container. In another example, multiple cell culture vessels may be serially connected to a single container, e.g., for the serial removal of spent growth medium from several cell culture vessels to a single waste container and/or for serial addition of fresh cell culture medium to several cell culture vessels.

Multiple sterile connections and liquid transfers to a cell culture vessel using multiple connection interfaces may involve multiple pairs of one connector and one container each. In such instances, methods may include moving a cell culture vessel to a first connection interface or vice versa, connecting a first connector to the cell culture vessel and a first container in the housing of the first connection interface, transferring liquid between the cell culture vessel and the first container, and repeating the moving, connecting, and transferring steps using additional connection interfaces, connectors, and containers until the desired number of liquid transfers has been performed.

For example, when using multiple connection interfaces for multiple sterile connections and liquid transfers, methods may include (a) moving a cell culture vessel to a first connection interface or vice versa, (b) connecting the first connector to the cell culture vessel and the first container in the housing of the first connection interface, (c) transferring liquid between the cell culture vessel and the first container, (d) moving a cell culture vessel to a second connection interface or vice versa, (e) connecting the second connector to the cell culture vessel and the second container in the housing of the second connection interface, (f) transferring liquid between the cell culture vessel and the second container, (g) moving a cell culture vessel to a third connection interface or vice versa, (h) connecting the third connector to the cell culture vessel and the third container in the housing of the third connection interface, and (i) transferring liquid between the cell culture vessel and the third container.

It should be appreciated that methods disclosed herein may be performed manually and/or by one or more automated devices such as robots or robotic arms. For example, the step(s) of moving the cell culture vessel to the connection interface(s) or vice versa may be performed by one or more robotic devices. In another example, the step(s) of connecting the cell culture vessel to the container(s) may be performed by one or more robotic devices. In yet another example, the cell culture vessel including a cell culture may be taken from an incubator arranged to house a plurality of cell culture vessels by one or more robotic devices.

Methods disclosed herein encompass sterilizing the sterilizable space after moving the cell culture vessel and/or the connection interface. The sterilization step may be performed prior to connecting the connector to the cell culture vessel and the container. Alternatively, or in addition to, the sterilization step may be performed after connecting the connector to the cell culture vessel and the container.

Any number of sterilization steps may be included in methods disclosed herein. For example, when methods include multiple sterile connections and liquid transfers, the sterilizable space may be sterilized prior to performing multiple sterile connections and liquid transfers or prior to performing each sterile connection and liquid transfer. In another example, when methods include multiple connection interfaces, the sterilizable space in each of the connection interfaces may be sterilized prior to performing a sterile connection and liquid transfer in that space.

A sterile connection and liquid transfer may be performed before and/or after processing of the cell culture in the cell culture vessel. For example, the cell culture vessel including the cell culture may be centrifuged and/or mixed prior to performing a sterile connection and liquid transfer. In another example, the cell culture vessel including the cell culture may be centrifuged and/or mixed after performing a sterile connection and liquid transfer. In yet another example, the cell culture vessel including the cell culture may be centrifuged and/or mixed both before and/or after performing a sterile connection and liquid transfer.

Methods disclosed herein encompass any moving of the cell culture vessel and the connection interface such that a sterile connection and liquid transfer may be performed. Accordingly, the moving step may involve moving the cell culture vessel to the connection interface or moving the connection interface to the cell culture vessel. Alternatively, or in addition to, the moving step may involve moving both the cell culture vessel and the connection interface.

Liquid transfer may be achieved using any suitable method for transferring liquids, e.g., transfer via gravity or a device such as a pump, vacuum, or pressurizer.

Connection interfaces and methods for sterile connection and liquid transfer described herein can be used for manufacturing cells, e.g., manufacturing immune cells expressing a chimeric receptor. Manufacturing cells may include culturing cells, expanding cells, or transducing cells. Manufacturing cells may involve any number of connection interfaces used to perform any number of sterile connections and liquid transfers.

Accordingly, methods disclosed herein may involve transferring cells and/or reagents for manufacturing cells, e.g., cell culture medium for growing cells and/or solutions including nucleic acids for transducing cells. For example, when manufacturing cells expressing a chimeric receptor, a solution including a nucleic acid coding for the chimeric receptor may be transferred from a container to a cell culture vessel for transducing the cells therein.

Such methods may use multiple containers to transfer cells and/or reagents into a cell culture vessel for manufacturing cells, e.g., for transducing cells. For example, the first container may include a cell culture medium for culturing cells, the second container may include a solution including a nucleic acid for transducing the cells, and the third container may be a destination bag for receiving either the cell culture medium or the cells.

Methods disclosed herein may also involve collecting the cells. For example, methods disclosed herein may result in collection of the cells in a container such as a destination bag. As such, methods disclosed herein may further include centrifuging a cell culture to obtain the collection of cells. Cells may be collected at any point during the manufacturing process, e.g., when transferring cells to a larger cell culture vessel during cell expansion or when harvesting cells for downstream processing or therapeutic use. Accordingly, cells may be collected in any one of the containers (e.g., the first, second, or third containers) used when performing multiple sterile connections and liquid transfers.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one of skill in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain, using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents, and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “including” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein in the specification and in the claims, the term “connected” is defined as attached, whether directly or indirectly through intervening components, and is not necessarily limited to physical connections. The connection can be such that the objects are permanently connected or releasably connected.

It should also be understood that, unless clearly indicated to the contrary, in any methods claimed herein that include more than one step or act, the order of the steps or acts of the method is not necessarily limited to the order in which the steps or acts of the method are recited.

Additional embodiments of the present disclosure are provided below for illustrative purposes only.

1. A method for making a sterile connection for transferring a liquid between at least two containers, comprising:
(a) positioning a first connection surface and a second connection surface, wherein the first connection surface is integral to a first connector and the second connection surface is integral to a second connector; and wherein the first connection surface is linked to a first container and the second connection surface is linked to a second container;
(b) activating a sterilization agent over the first connection surface and the second connection surface within a sterilization chamber to produce a sterile environment;
(c) fluidically coupling the first container with the second container through the first connection surface and the second connection surface;
(d) transferring a liquid between the first container and the second container; and optionally
(e) disconnecting the first connection surface and the second connection surface after step (d).

In a preferred embodiment step (a) comprises forming a gap between the first connection surface and the second connection surface via partial coupling of the first and second connectors, and step (b) comprises activating the sterilization agent within the gap.

2. The method of item 1, wherein the first and second connectors are loaded into a sterilization chamber or housing.

3. The method of item 1 or item 2, wherein step (a) comprises forming a gap between the first connection surface and the second connection surface via partial coupling of the first and second connectors, and wherein step (b) comprises activating the sterilization agent within the gap.

4. The method of any one of items 1-3, wherein the sterilization agent is a fluid, gas, vapor, or steam, and activating the sterilization agent comprises flowing the sterilization agent through the gap.

5. The method of any one of items 1-3, wherein step (b) comprises activating an energy source within the chamber, the energy source selected from one of an ultra-violet radiation, e-beams, an X-ray, a gamma ray, an ultrasonic beam, or heat.

6. The method of any one of items 1-3, wherein the sterilization agent is a fluid, and activating the sterilization agent comprises flowing the fluid through at least a portion of the chamber.

7. The method of any one of items 1-6, wherein activating the sterilization agent over the first connector and the second connector is performed prior to fluidically coupling the first container with the second container.

8. The method of any one of items 1-7, wherein step (c) comprises fluidically coupling the first and second container with a fluid transfer conduit, the fluid transfer conduit having connectors to couple either end of the fluid transfer conduit with to the first connector and the second connector.

9. The method of any one of items 1-8, wherein the first container comprises a cell culture medium and the second container comprises cultured cells, and wherein transferring a liquid between the first container and the second container comprises transferring the cell culture medium to the cultured cells.

10. The method of any one of items 1-9, wherein the first connector and the second connector are connected via a third connector such that the first connector connects to one end of the third connector and the second connector connects to the other end of the third connector.

11. The method of item 10, wherein the intermediate connector is sealable for removal of samples from the fluid flow path.

12. The method of any one of items 1-11, wherein transferring a liquid between the first container and the second container in step (d) comprises interlocking one or more pinch valves and/or pumps.

13. The method of any one of items 1-11, wherein transferring a liquid between the first container and the second container in step (d) comprises rotating a peristaltic pump to cause a positive or a negative pressure in a tubing between the first container and the second container.

14. The method of any one of items 1-11, wherein transferring a liquid between the first container and the second container in step (d) comprises rotating a peristaltic pump between two interlocking valves prior to activating the interlocking valves.

15. The method of any one of items 1-14, wherein the first container comprises one or more source bags containing a culture medium or a waste medium, and the second container comprises one or more culture bags containing a cell culture.

16. The method of any one of items 15, wherein transferring a liquid between the first container and the second container in step (d) comprises transferring a solution including a nucleic acid or a viral particle comprising such to the second container for transducing a plurality of cells therein, optionally wherein the nucleic acid encodes a chimeric receptor.

17. The method of any one of items 1-14, wherein the first container is a cell culture vessel, the second container is a designation bag, and transferring a liquid between the first container and the second container in step (d) comprises transferring a culture medium or a plurality of cells into the designation bag from a cell culture vessel.

18. The method of any one of items 1-17, wherein the second container is a cell culture vessel, and wherein the method further comprises connecting a third container and the cell culture vessel, and transferring liquid between the cell culture vessel and the third container.

19. The method of any one of items 2-18, wherein positioning the first connection surface and the second connection surface in step (a) comprises robotically loading the first connector and the second connector within the chamber.

20. The method of any one of items 1-19, wherein transferring a liquid between the first container and the second container in step (d) comprises pumping the liquid from the first container to the second container.

21. The method of any one of items 1-10, wherein the second container comprises a cell culture vessel, and the method further comprising collecting a cell culture from an incubator housing a plurality of cell culture vessels.

22. The method of any one of items 1-21, wherein the first container or the second container is a destination bag comprising a plurality of cells.

23. The method of item 22, wherein the cells are immune cells, which optionally are T cells.

24. The method of item 23, wherein the immune cells express a chimeric receptor.

25. A device, comprising:
(i) holders for positioning a first connection surface and a second connection surface, wherein the first connection surface is integral to a first connector and the second connection surface is integral to a second connection; and wherein: the first connection surface is linked to a first container and the second connection surface is linked to a second container;
(ii) a pump configured to transfer a liquid from the first container to the second container; and
(iii) a sterilization agent.

26. The device of item 25, further comprising (iv) an interlock valve configured to avoid back contamination between the first container and the second container.

27. The device of item 25 or item 26, further comprising (v) a first controller configured to activate the sterilization agent over the first connector and the second connector.

28. The device of any one of items 25-27, further comprising (vi) a second controller configured to position the first connector and the second connector for sterilization and/or to remove the first connector or the second connector from the sterilization position after liquid transfer between the first container and the second container.

29. The device of any one of items 25-28, further comprising (vii) a peristaltic pump between the first connector and the second connector to prevent backflow between the first container and the second container.

30. The device of any one of items 25-29, further comprising (viii) one or more interlocking pinch valves between the first container and the second container to prevent backflow between the first container and the second container.

31. A connection interface for sterile connection and liquid transfer, wherein the connection interface comprises:
a first connector;
a second connector;
a first sterilizable space, which optionally comprises a sterilization agent, wherein the first connector is fluidically coupled with a first container and the second connector is fluidically coupled with a second container in the first sterilizable space; and
a liquid transfer device including pumps and valves to allow for liquid transfer between the first container and the second container and/to avoid back contamination.

32. The connection interface of item 31, wherein the first container includes a solution for transferring into a cell culture vessel.

33. The connection interface of any one of items 31 and 32, wherein the second container comprises a cell culture vessel.

34. The connection interface of any one of items 31-33, wherein a solution in the first container is a culture medium for culturing cells grown in the second container.

35. The connection interface of any one of items 31-34, wherein a solution in the first container comprises a nucleic acid or a viral particle comprising such for transducing cells grown in the second container, and wherein the nucleic acid encodes a chimeric receptor.

36. The connection interface of any one of items 31-35, wherein the second container comprises a cell culture and the first container is a destination bag for receiving either a culture medium or multiple cells in the cell culture.

37. The connection interface of any one of items 31-36, wherein the first container comprises a cell culture medium or a viral vector for transferring into the second container, which comprises a first cell culture, wherein the connection interface further comprises a third container including a second cell culture configured to receive the cell culture medium or the viral vector from the first container.

38. The connection interface of any one of items 31-37, wherein the second container comprises a destination bag for receiving either a culture medium or multiple cells from a cell culture.

39. The connection interface of any one of items 31-38, wherein the sterilizer agent comprises an energy source selected from the group consisting of UV light, e-beams, gamma rays, heat, and steam.

40. The connection interface of any one of items 31-39, wherein the sterilizer agent comprises a fluid selected from a gas or a vapor.

41. The connection interface of any one of items 31-40, wherein the connection interface further comprises a pump for liquid transfer between the second container and the first container, a second container, and/or the third container.

42. The connection interface of any one of items 31-41, wherein the first connector, the second connector, and/or the third connector is removable, disposable, reusable, or a combination thereof.

43. The connection interface of any one of items 31-42, wherein the first connector, the second connector, and/or the third connector comprises a septum and/or a cannula.

44. The connection interface of any one of items 31-43, wherein the first connector, the second connector, and/or the third connector is ejectable from the connection interface.

45. The connection interface of any one of items 31-44, wherein the first container, the second container, and/or the third container comprises a fluid conduit, and the first connector, the second connector, and/or the third connector is arranged to be attached to the fluid conduit.

46. The connection interface of any one of items 31-45, wherein the second container comprises a septum, and the first connector, the second connector, and/or the third connector is arranged to be attached to the septum.

47. The connection interface of any one of items 31-46, wherein:
the first connector, and/or the second connector each comprise a first piece and a second piece,
one end of the first piece being arranged to be attached to the first container, a second container, and/or the third container, and
one end of the second piece being arranged to be attached to the second container.

48. The connection interface of any one of items 31-47, wherein the first connector or the second connector comprise a first piece and a second piece, further comprising an intermediate piece having a first end and a second end, which are arranged to be attached to a second end of the first piece and/or a second end of the second piece.

49. The connection interface of any one of items 31-48, wherein the first connector or the second connector each comprise a first piece and a second piece,
the first container and the second container comprises a fluid conduit, and the first piece is arranged to be attached to the fluid conduit, and
the second container comprises a septum, and the second piece is arranged to be attached to the septum.

50. The connection interface of any one of items 31-49, wherein the first connector or the second connector comprise a first piece and a second piece, wherein the first piece and the second piece form the first sterilizable space, a second sterilizable space, and/or a third sterilizable space.

51. The connection interface of any one of items 31-49, wherein:
the first connector or the second connector comprise a first piece and a second piece,
the first piece and the second piece comprise: one or more valves, one or more seals, and one or more ports.

1. A method for making a sterile connection for transferring a liquid between at least two containers, comprising:
(a) positioning a first connection surface and a second connection surface, wherein the first connection surface is integral to a first connector and the second connection surface is integral to a second connector; and wherein the first connection surface is linked to a first container and the second connection surface is linked to a second container;
(b) coupling the first connector and the second connector, wherein the coupling defines a sterilization chamber comprising a gap between the first connection surface and the second connection surface;
(c) activating a sterilization agent within the sterilization chamber to produce a sterile environment;
(d) fluidically coupling the first container with the second container through the first connection surface and the second connection surface;
(e) transferring a liquid between the first container and the second container; and optionally,
(f) disconnecting the first connection surface and the second connection surface after step (e).

2. The method of claim 1, wherein the gap between the connection surfaces is a closed space accessible through at least one opening, optionally a port.

3. The method of claim 2, wherein
(i) the sterilization agent is a fluid, gas, vapor, or steam; and
(ii) activating the sterilization agent comprises flowing the sterilization agent through the gap between the connection surfaces through the ports.

4. The method of claim 1, wherein the first and second connectors are loaded into a housing, optionally wherein any of the steps (a)-(f) or in combination is operated automatically by a controller.

5. The method of claim 1, wherein the first and second connectors are loaded into a housing, optionally and wherein positioning the first connection surface and the second connection surface in step (a) comprises robotically loading the first connector and the second connector within the housing.

6. The method of claim 1, wherein
(i) activating the sterilization agent over the first connector and the second connector is performed prior to fluidically coupling the first container with the second container;
(ii) step (d) comprises fluidically coupling the first and second container with a fluid transfer conduit, wherein the fluid transfer conduit has connectors to couple either end of the fluid transfer conduit with to the first connector and the second connector;
(iii) the first container comprises a cell culture medium and the second container comprises cultured cells, and wherein transferring a liquid between the first container and the second container comprises transferring the cell culture medium to the cultured cells; and/or
(iv) the first connector and the second connector are connected via a third connector such that the first connector connects to one end of the third connector and the second connector connects to the other end of the third connector, optionally wherein the intermediate connector is sealable for removal of samples from the fluid flow path.

7. The method of claim 1, wherein
(i) transferring a liquid between the first container and the second container in step (e) comprises interlocking one or more pinch valves and/or pumps;
(ii) transferring a liquid between the first container and the second container in step (e) comprises rotating a peristaltic pump to cause a positive or a negative pressure in a tubing between the first container and the second container; or
(iii) transferring a liquid between the first container and the second container in step (e) comprises rotating a peristaltic pump between two interlocking valves prior to activating the interlocking valves.

8. The method of claim 1, wherein
(i) the first container comprises one or more source bags containing a culture medium or a waste medium, and the second container comprises one or more culture bags containing a cell culture, optionally wherein transferring a liquid between the first container and the second container in step (e) comprises transferring a solution including a nucleic acid or a viral particle comprising such to the second container for transducing a plurality of cells therein, optionally wherein the nucleic acid encodes a chimeric receptor; or
(ii) the first container is a cell culture vessel, the second container is a designation bag, and transferring a liquid between the first container and the second container in step (e) comprises transferring a culture medium or a plurality of cells into the designation bag from a cell culture vessel.

9. The method of claim 1, wherein the second container is a cell culture vessel, and wherein the method further comprises connecting a third container and the cell culture vessel, and transferring liquid between the cell culture vessel and the third container.

10. The method of claim 1, wherein transferring a liquid between the first container and the second container in step (e) comprises pumping the liquid from the first container to the second container.

11. The method of claim 1, wherein the second container comprises a cell culture vessel, and the method further comprising collecting a cell culture from an incubator housing a plurality of cell culture vessels.

12. The method of claim 1, wherein the first container or the second container is a destination bag comprising a plurality of cells, preferably wherein the cells are immune cells, which optionally are T cells, more optionally wherein the immune cells express a chimeric receptor.

13. A device, comprising:
(i) holders for positioning a first connection surface and a second connection surface, wherein the first connection surface is integral to a first connector and the second connection surface is integral to a second connection; and wherein the first connection surface is linked to a first container and the second connection surface is linked to a second container;
(ii) the first connector and the second connector configured to form a sterilization chamber comprising a gap between the first connection surface and the second connection surface;
(iii) a pump configured to transfer a liquid from the first container to the second container; and
(iv) a sterilization agent that is activatable within the gap between the first connection surface and the second connection surface,
preferably, wherein the gap is an enclosed space accessible through at least one opening, preferably optionally a port.

14. The device of claim 13, wherein the first connector and/or the second connector comprises ports suitable to provide access for the sterilization agent to the gap.

15. The device of claim 13, further comprising:
(iv) an interlock valve configured to avoid back contamination between the first container and the second container;
(v) a first controller configured to activate the sterilization agent over the first connector and the second connector;
(vi) a second controller configured to position the first connector and the second connector for sterilization and/or to remove the first connector or the second connector from the sterilization position after liquid transfer between the first container and the second container;
(vii) a peristaltic pump between the first connector and the second connector to prevent backflow between the first container and the second container; and/or
(viii) one or more interlocking pinch valve between the first container and the second container to prevent backflow between the first container and the second container.

16. The device of claim 13, wherein the device contains a controller configured to automatically operate any of the steps (i)-(viii) or in combination.

17. A connection interface for sterile connection and liquid transfer, wherein the connection interface comprises:
a first connector;
a second connector;
wherein the first connector and the second connector define a sterilization chamber comprising a gap between the first connection surface and the second connection surface;
preferably, wherein the gap is an enclosed space accessible through at least one opening, preferably optionally a port;
wherein the gap optionally comprises a sterilization agent;
wherein the first connector is fluidically coupled with a first container and the second connector is fluidically coupled with a second container; and
a liquid transfer device including pumps and valves to allow for liquid transfer between the first container and the second container and/to avoid back contamination.

18. The connection interface of claim 17, wherein
(i) the first container includes a solution for transferring into a cell culture vessel;
(ii) the second container comprises a cell culture vessel;
(iii) a solution in the first container is a culture medium for culturing cells grown in the second container;
(iv) a solution in the first container comprises a nucleic acid or a viral particle comprising such for transducing cells grown in the second container, and wherein the nucleic acid encodes a chimeric receptor;
(v) the second container comprises a cell culture and the first container is a destination bag for receiving either a culture medium or multiple cells in the cell culture;
(vi) the first container comprises a cell culture medium or a viral vector for transferring into the second container, which comprises a first cell culture, wherein the connection interface further comprises a third container including a second cell culture configured to receive the cell culture medium or the viral vector from the first container;
(vii) the second container comprises a destination bag for receiving either a culture medium or multiple cells from a cell culture;
(viii) the sterilization agent comprises a fluid, a gas, or a vapor;
(ix) the connection interface further comprises a pump for liquid transfer between the second container and the first container, a second container, and/or the third container;
(x) the first connector, the second connector, and/or the third connector is removable, disposable, reusable, or a combination thereof;
(xi) the first connector, the second connector, and/or the third connector comprises a septum and/or a cannula;
(xii) the first connector, the second connector, and/or the third connector is ejectable from the connection interface;
(xiii) the first container, the second container, and/or the third container comprises a fluid conduit, and the first connector, the second connector, and/or the third connector is arranged to be attached to the fluid conduit;
(xiv) the second container comprises a septum, and the first connector, the second connector, and/or the third connector is arranged to be attached to the septum;
(xv) the first connector, and/or the second connector each comprise a first piece and a second piece,
one end of the first piece being arranged to be attached to the first container, a second container, and/or the third container, and
one end of the second piece being arranged to be attached to the second container;
(xvi) the first connector or the second connector comprise a first piece and a second piece, further comprising an intermediate piece having a first end and a second end, which are arranged to be attached to a second end of the first piece and/or a second end of the second piece;
(xvii) the first connector or the second connector each comprise a first piece and a second piece,
the first container and the second container comprise a fluid conduit, and the first piece is arranged to be attached to the fluid conduit, and
the second container comprises a septum, and the second piece is arranged to be attached to the septum; and/or
(xviii) the first connector or the second connector comprise a first piece and a second piece, wherein the first piece and the second piece form the first sterilizable space, a second sterilizable space, and/or a third sterilizable space; or the first connector or the second connector comprise a first piece and a second piece, the first piece and the second piece comprise: one or more valves, one or more seals, and one or more ports.
